(19) United States
(12) Patent Application Publication
(10) Pub. No.: US 2006/0205700 A1
(43) Pub. Date: Sep. 14, 2006

(54) Non invasive method for prevention and treatment of cancer

(76) Inventors: Flavia S. May, Cambridge, MA (US); Michael G. May, Westlake Village, CA (US)

Correspondence Address: RONALD E. GREIGG;GREIGG & GREIGG P.L.L.C., 1423 POWHATAN STREET, UNIT ONE, ALEXANDRIA, VA, 22314, US

(21) Appl. No.: 11/074,757

(22) Filed: Mar. 9, 2005

(51) Int. Cl. A61K 31/56

(52) U.S. Cl. 514/169

(57) ABSTRACT

This invention relates to a novel non invasive method for preventing or treating cancer or its recurrence and to the novel use of compounds useful therein. Very low cost, rapid action and generally no side effects are also objects inherent to the invention, realized through novel use of otherwise well known and mass produced compounds, which disturb, block and interrupt intra cellular signaling pathways, triggering concurrently inhibition of vital survival and growth factors produced by cancer cells.

### BACKGROUND OF THE INVENTION

The invention pertains to a method for non invasive prevention and treatment of cancer for human beings. The prior art of non invasive anticancer therapeutics are targeting specific expressions or characteristics of cancers as for example excessive proliferation, negation of apoptosis, promotion of angiogenesis and drug resistance.

The various approaches comprise monoclonal antibodies, recombinant proteins, antisense and RNAi technology, small molecules or tumor vaccines. These target based therapies mainly focus on the interruption of signal pathways involved in the formation of cancer cell phenotypes. Due to the robustness and adaptability of cancer cells with their complex signaling cascades, providing them with redundant, overlapping and intersecting networks, these therapeutics have shown limited effects, extending median survival by some months only.

Given the complexity of cancer biology, targeting any single signaling element of the complex network did not produce relevant tumor response.

### SUMMARY OF THE INVENTION

Based on the foregoing, it is an object of the present invention to provide for a method for non invasive prevention and treatment of cancer for human beings with a high rate of success and significant increase of the median survival time.

This object is attained with a method, wherein an amount of a compound is administered thereto which is effective to inhibit dysfunctions of genes of the cancer cell, said compound being a glucocorticoid antagonist binding competitively to related receptors by having strong affinity to glucocorticoid receptors, without activating them.

### DESCRIPTION OF THE INVENTION

In the following the term compound is used for competitive glucocorticoid receptor antagonists, preferably those having a low agonist and a high abortifacient activity. Such compounds and useful variations thereof are disclosed e.g. in the U.S. Pat. No. 6,608,074.

The present invention is the consequence of thorough studies of the key factors governing the life of cancer cells and originates out of two different approaches.

First

Cancer cells have an intelligent survival and growth strategy. This strategy comprises complex signaling cascades enabling the cells to produce messenger molecules, markers, enzymes and signaling factors, governing cell adhesion, proliferation, malign neovascularization, suppression of apoptosis, immune escape, invasiveness and drug resistance, which are essential characteristics or properties, respectively expressions of cancer.

These characteristics are vital for survival and growth of cancer. They depend on signaling cascades which are triggered by stereoids acting on receptors related to genes which let the cell produce the proteins responsible for the characteristics.

Surprising is the absence of a weighting of the vital survival requirements of all types of cancer cells. The various steps leading to a single cancer cell are reasonably well understood, but single abnormal cells either undergo apoptosis or are killed and evacuated by the immune system of the related body. Therefore the very first and most important characterristic of malign cells is the ability to hide from the immune system, the immune escape.

According to the invention, this task is intelligently accomplished by the cancer, pretending to be a desired entity starting to grow. The growth hormones and human chorionic gonadotropin imitate and simulate embryonal cells and enhance implantation and growing.

Said growth and gonadotropin hormones are, according to the invention, the key factors in the immuno resistance and they efficiently subvert the immune system by simulating the growth of an embryo. Also the implantation and malign neovascularization of the cancer in the epithelium or endothelium are essentially controlled by the same hormones. By inhibiting the formation of epithelium growth and endothelium growth, the hiding, implantation and growth of cancer cells is rendered impossible.

The growth hormones generated by the growth factor expressing gene of the cancer cell also impedes the cellular death and enhances proliferation and invasiveness and metastatic spread.

In summary, all vital survival and growth conditions generated by the dysfunctional genes and their controlling signaling cascades are linked altogether to the same glucocorticosteroid hormones and therefore are subject to an equally acting or concurrently operating inhibition by the action of a competitive glucocorticoid receptor antagonist. After this scenario of blocking the cell receptors is initiated, the immune system can again become active in assisting the destruction and elimination of the now disguised cancer cells.

According to the aforesaid, cancer can also be described as malign dysfunction of genes. If the glucocorticoid receptors of the cancer cells are occupied by competitive glucocorticoid receptor antagonists, the whole related signaling cascades are inhibited. By administering a therapeutically effective amount of competitive glucocorticoid antagonists, inhibition of metabolic dysfunctions of cancer cells, essentially those dysfunctions related to expressions of glucocorticoid receptor regulated genes, is obtained.

The dysfunctional production of the vital agents by cancer cells—mainly the growth hormones—is inhibited by occupying the related receptors with antagonists. The malign dysfunction of genes, their related signaling receptors and their hormone production is according to the invention inhibited by glucocorticoid antagonists.

While several facts resulting in the failure of the control system that regulate normal cell division are well known, the main causes for immune escape of cancer has not previously been associated with dysfunctions of glucocorticoid receptor regulated genes.

Second

Seen now from a different point of view, U.S. Pat. No. 6,608,074 teaches the use of competitive progesterone antagonists for inhibiting the formation of endometrial glands and epithelium growth, thereby rendering the implantation of a fertilized egg in the uterus impossible. The active component of the compound according to U.S. Pat. No. 6,608,074 can displace progesterones from their receptors. This active component is mentioned also as useful for therapeutic termination of pregnancy and also as a glucocorticoid antagonist for the treatment of Cushing's syndrome. According to the present invention, the abortive action of the above mentioned active components or improvements thereof are actively and equally acting or concurrently generating inhibitive actions against the various vital survival and growth allowing factors generated by cancer cells.

As is well known tumors and adenomas are sometimes preliminary stages of cancer. In a preventive manner the inhibition of their growth can therefore be achieved by the inventive use of the same compounds.

Smaller dosages and wider time spacing are indicated in the case of prevention of any tumoral growth or it's recurrence. Similarly to applications of known compounds, the compound can be administered orally or parenterally as well as by nasal application, rectally or sublingually in a dosage range from about 5 to about 2000 mg on a regimen in single or 2 to 4 devided daily doses. Even smaller values like 50-100 mg are senseful for antirecurrence and preventive applications as such doses already show implantation inhibition effects.

The active substance can be utilized in a composition such as tablet, capsule, solution, suspension or other known ways and as called for by accepted medical and/or pharmaceutical practice.

1. A method for non invasive prevention and treatment of cancer for human beings, wherein an amount of a compound is administered thereto which is effective to inhibit dysfunctions of genes of the cancer cell, said compound being a glucocorticoid antagonist binding competitively to the receptors related to and triggering said dysfunctional genes.

2. A method according to claim 1, wherein the glucocorticoid antagonist is effective for inhibiting the interrelated malign signaling cascades responsible for: immune escape,
anti apoptosis,
angiogenesis,
proliferation,
invasiveness, and
cell adhesion.

3. A method according to claim 1, wherein the receptors acting on the genes responsible for the production of human chorionic gonadotropin and growth hormones are occupied by glucocorticoid antagonist molecules.

4. A method according to claim 1, wherein the receptors acting on the genes responsible for the production of progesterones by the cancer are occupied by glucocorticoid antagonist molecules.

5. A method for non invasive prevention and treatment of cancer for human beings, wherein an amount of a compound is administered thereto which is effective to inhibit the cancer cell's hormone production cascades responsible for the immune escape and proliferation, by occupying the related receptors of the responsible genes, said compound being a glucocorticoid antagonist.

6. A method for the non invasive prevention or treatment of cancer, comprising administration to a person in need or desire of treatment a therapeutically effective amount of the compound defined in claim 1 or 2.

7. A method for non invasive prevention and treatment of cancer for human beings, wherein the body's defense system is enabled to detect and destroy the cancerous cells by inhibiting their immune escape mechanism by administering a pharmaceutically appropriate amount of a glucocorticoid antagonist.

8. Use of a compound as defined in claim 1, said compound inhibiting formation of epithelial and epidermal growth, thereby inhibiting implantation and support of cancer cells.

9. Use of a glucocorticoid antagonists according to claim 1, for inhibiting the formation of stroma—the sustaining tissue of cancer.

10. Use of a glucocorticoid antagonists according to claim 1, for inhibiting the otherwise continuous production of growth hormones by cancer cells.
